(19) United States
(12) Patent Application Publication
(10) Pub. No.: US 2005/0075274 A1
(43) Pub. Date: Apr. 7, 2005

(54) Method for simulating the interaction of chemical compounds with live organisms

(75) Inventors: Stefan Willmann, Dusseidorf (DE); Walter Schmitt, Neuss (DE); Franco Fois, Monheim (DE)

Correspondence Address: NORRIS, McLAUGHLIN & MARCUS, P.A., 18th Floor, 875 Third Avenue, New York, NY, 10022, US

(73) Assignee: Bayer Technology Services GmbH, Leverkusen (DE)

(21) Appl. No.: 10/936,353

(22) Filed: Sep. 8, 2004

(30) Foreign Application Priority Data
Oct. 2, 2003 (DE) 103 45 836.0

(51) Int. Cl. A61K 31/00; G06F 19/00; G01N 33/48; G01N 33/50

(52) U.S. Cl. 514/1; 702/19; 600/300

(57) ABSTRACT

A method for determining the interaction of one or more chemical active compounds with organisms. The method comprises selecting at least one compound and analyzing its physiko-chemical and/or biochemical characteristics including its pharmacokinetic properties in various anatomical and physiological compartments of the organism in question. The relevant physiological compartments are characterized with respect to the compound's various pharmacokinetic properties that may vary with the mode of administering the compound. A combined system of coupled differential equations is then used to simulate a concentration-time curve of the active compound in selected compartments.

The invention relates to a computer program for calculating the pharmacokinetic and pharmacodynamic behaviour of chemical compounds in live organisms, for example in mammals, insects or plants. The rates and extents of uptake, distribution, metabolism and excretion of chemical compounds in live organisms are of great importance, for example with regard to the in vivo activity of pharmaceutical active compounds and crop protection agents, or for the toxicological risk assessment. Computer programs such as the physiologically-based pharmacokinetic (PB-PK) and pharmacodynamic (PD) modelling are suitable for describing interactive processes of chemical compounds with live organisms. The invention makes possible the particularly simple generation of such models based on a multi-level architecture in which individually encapsulated modules which describe, for example, the organism, the substance, the type of application or the type of activity, are interconnected dynamically. By linking these modules to an integrated model, a system of coupled differential equations (mass conservation equations) is generated and solved numerically by a solver. The particular advantage of the invention is the high flexibility of the modular computer program, owing to which a multiplicity of complex physiological and biochemical scenarios can be generated in a particularly simple manner. The chemical, biological, physiological and anatomical input parameters which are required for generating this equation system can either be present in one or more relational databases or input via a graphical user interface.

A large number of PB-PK and PD models have been described in the literature for a variety of organisms such as mammals [R. Kawai, M. Lemaire, J. L. Steimer, A. Bruelisauer, W. Niederberger, M. Rowland: Physiologically based pharmacokinetic study on a Cyclosporin derivative, SDZ IMM 125. J. Pharmacokin. Biopharm. 22, 327-365,(1994); P. Poulin, F. P. Theil: Prediction of pharmacokinetics prior to in vivo studies. 1. Mechanism-based prediction of volume of distribution. J. Pharm. Sci. 291, 129-156, (2002); P. Poulin, F. P. Theil: Prediction of Pharmacokinetics prior to in vivo studies. II. Generic physiologically based pharmacokinetic models of drug disposition, J. Pharm. Sci. 91, 1358-1370 (2002)], Insects [R. Greenwood, M. G. Ford, E. A. Peace, D. W. Salt: The Kinetics of Insecticide Action. Part IV: The in vivo Distribution of Pyrethroid Insecticides during Insect Poisoning. Pestic. Sci. 30, 97-121 (1990)] or plants [N. M. Satchivi, E. W. Stoller, L. M. Wax, D. P. Briskin: A nonlinear dynamic simulation model for xenobiotic transport and whole plant allocation following foliar application. Pestic. Biochem. Physiol. 68, 67-84 (2000); N. M. Satchivi, E. W. Stoller, L. M. Wax, D. P. Briskin: A nonlinear dynamic simulation model for xenobiotic transport and whole plant allocation following foliar application. Pestic. Biochem. Physiol. 68, 67-84 (2000)]. Such models are successfully employed to describe the behaviour of chemical compounds on the basis of physiological and anatomical information and substance-specific physico-chemical and biochemical parameters. The current procedure is to divide the organism to be described into several physiological compartments. Frequently, individual organs such as the liver, muscles or the lungs correspond to these compartments. These compartments are characterized by physiological parameters such as, for example, blood flow rates and by their water, fat and protein contents [R. Greenwood, M. G. Ford, E. A. Peace, D. W. Salt: The Kinetics of Insecticide Action. Part IV: The in vivo Distribution of Pyrethroid Insecticides during Insect Poisoning. Pestic. Sci. 30, 97-121 (1990)]. To obtain a physiologically-based model, these compartments are linked to one another numerically by mass conservation equations to match the anatomical parameters of the organism, as is illustrated in detail in the above-cited prior art.

In accordance with the prior art, this mathematical coupling is rigid, i.e. the differential equations are hard-wired to one another. This means that a model, once generated, remains fixed with regard to the number of its compartments and their physiological circuitry. The invention is based on the object of developing a flexible method which dynamically interconnects a variable number of compartments and modules (for example regarding the organism, the substance or the type of application) in accordance with individual parameters, and automatically generates, and subsequently solves numerically, the corresponding physiologically-based model.

This technical problem is solved by a novel modular programming method. The method is based on a library of modules which are independent of one another, are closed within themselves and have different functionalities (for example the definition of substance characteristics or the type of application, or the description of the physiology of the organism to be studied, and the like). It is only during the execution time of the program that these modules are combined dynamically to give a complete model. The method described herein thus permits for the first time the flexible treatment of different complex scenarios without the necessity of carrying out changes at the level of the differential equations.

Modular programming concepts are known in principle from other fields of application (see, for example, U.S. Pat. No. 5,930,154 or WO 00/65523), but they were hitherto not employed for pharmacokinetic or pharmacodynamic problems.

The subject matter of the invention, by means of which the abovementioned problem is solved, is a method for determining the interaction of one or more chemical active compounds with organisms, comprising the following steps:
A) selection of at least one substance and analysis of its physiko-chemical and/or biochemical characteristics, in particular from the series consisting of lipophilicity, solubility, protein binding, molecule size (expressed as molecular weight or volume), pKa value in the case of acids or bases, metabolic degradation rate and kinetic constants of active transporters,
B) selection of anatomical and physiological compartments of the organism in question, selected from at least amongst: a) in the case of mammals or insects: the lungs, peripheral organs, blood vessels, preferably arteries and/or veins, or blood fluid; b) in the case of plants: xylem and phloem flow, and root and/or leaf and/or stem
C) description of the compartments in a computing program with regard to the transport processes of the substance from and to the compartments, degradation processes and active processes by mass transportation equations and kinetic reaction equations for describing the process in question
D) description of the transport processes and, if appropriate, the degradation processes of the active compound of the organism, in particular in the case of intravenous, peroral, inhalative or subcutaneous administration, in the computing program by mass transportation equations and kinetic reaction equations,
E) combination of the equation systems selected in steps C) and D) and, in particular, numerical calculation of the resulting system of coupled differential equations,
F) determination of the concentration-time curve of the active compound in selected compartments.

The organism(s) represent(s) preferably mammals from the group consisting of man, monkey, dog, pig, rat or mouse, or insects, in particular caterpillars, or plants.

In accordance with a preferred embodiment, the active compound is applied via the intravenous, oral, topical, subcutaneous, intraperitoneal route, the inhalative route via the nose or the lungs, or the intracerebrovascular route (in the case of mammals), or via the intrahaemolymphatic, topical or oral route via feeding or gavage (in the case of insects), or else in the form of a spray mixture with foliar uptake or uptake via the roots (in the case of plants).

Preferred is a method which is characterized in that the physiological parameters which describe the organisms are time-dependent parameters.

In a preferred embodiment of the method, some of the parameters will be taken from a database which is linked to the computer system.

In a preferred embodiment, the physiological and anatomical parameters can be varied within a predetermined statistical variation, using random numbers (population kinetics).

Especially preferred is a method in which a hierarchical model architecture consisting of modules with different functionalities, and at least two modules which comprise either chemical or biological characteristics of the substance or which comprise physiological or anatomical information on the organism or which comprise information on the mode of application or the type of activity are used for the computing program.

An especially preferred variant of the method is characterized in that a multiplicity of models of organisms are combined in an integrated model, in particular for the simulation of drug-drug interactions, mother-foetus model, combined insect-plant model.

The core of the present invention is the realization of a modular simulation concept with dynamic generation of a differential equation (DEQ) system and its application to pharmacokinetic and pharmacodynamic problems. The simulation kernel for this purposes consists of a library of individual modules which comprise the following functionalities:
definition of the physico-chemical compound characteristics (for example lipophilicity, affinity to plasma proteins, molecular weight)
anatomical and physiological description of the organism (mammal, insect, plant)
description of the individual compartments (organs) which form the organism
description of the type of application of the chemical compound (for example via the intravenous, oral, subcutaneous, topical or inhalative route for mammals, the oral or topical route for insects, application as a spray mixture and foliar uptake or uptake via the roots for plants)
time loop definition for numeric integration
numeric integration of the differential equation system (for example by the method of Euler or Runge-Kutta)
storage of the simulation results (for example in a file, database or the like).

These individual modules are interconnected dynamically in a hierarchical, predefined manner via a computer system, for example a commercially available PC, during the execution time of the program. Thereafter, a system of coupled DEQs (mass conservation equations) is generated automatically and solved by numeric integration. As a result, the model provides concentration-time curves of the respective chemical compound in the diverse compartments of the organism in question, and these curves can then be used for other purposes, for example for calculating a pharmacological activity.

The minimal model on which the invention is based consists of at least one substance module, an application module, an organism module, the DEQ builder and the numerical solver for the DEQ system, and optional further modules, for example a module for pharmacodynamic activity.

The substance module comprises physico-chemical and/or biochemical information on the substance whose behaviour is to be simulated. This information can be, for example, values for the lipophilicity, solubility, for example water or intestinal fluid, protein binding, for example of plasma proteins, molecule size (expressed as molecular weight or volume), pKa value in the case of acids or bases, metabolic degradation rates, kinetic constants of active transporters and the like. These parameters can generally be determined by in vitro experiments or—in individual cases, for example in the case of lipophilicity—calculated directly from the structure by means of known forecasting models which employ, for example, QSAR, HQSAR. or neuronal networks.

The organism module comprises physiological and anatomical information which characterizes the organism with which the substance is to interact. Organism-specific information is, inter alia, volumes and volume blood flow rates, and the water, fat and protein contents of the individual compartments (in the case of mammals and insects) or xylem and phloem transport rates and the volumes of symplast, apoplast and vacuoles in the case of plants. These physiological parameters can be constant in time or else a function of time, for example in order to be able to take into consideration growth processes and other physiological changes over time. These physiological and anatomical parameters are published in the literature for a multiplicity of relevant organisms.

The application module comprises information on the location and the time profile of the administration of the substance. Conventional forms for the administration of pharmaceutically active compounds in mammals are the intravenous application in the form of a bolus or an infusion, the oral application in the form of a solution, capsule or tablet, the subcutaneous administration, the intraperitoneal administration, the topical administration with uptake via the skin or the mucous membranes, and the nasal or inhalative administration. In insects, the uptake of chemical compounds is predominantly oral or via the cuticle following topical contact. The uptake of substances in plants is effected via the roots or via the leaves.

The DEQ builder automatically generates the differential equation system based on the mass balance within the organism. The solver deals with the numeric integration of the DEQ system and gives concentration/time profiles of the chemical compound in the compartments of the organism as the result. These data can be utilized further for example for describing a pharmacological effect at the target enzyme.

To link the modules in a dynamic fashion, a hierarchical management of the variables comprised in the modules is required since various variables from different modules can interdepend. Thus, for example, the substance-specific physico-chemical parameters (from the substance module) are used together with physiological information (from the organism module) for calculating equilibrium distribution coefficients between the plasma and the peripheral compartments in the mammalian body. Similarly, dependencies exist between the individual compartments within one organism. For example, the blood flow in the lungs is the result of the sum of blood flow rates of all the remaining organs since blood circulation in the mammalian body is a closed system. Such dependencies require a general hierarchical data structure so that they can be recognized automatically and taken into consideration. Such a hierarchical data structure is part of the present invention. To this end, the modules are administered from a database of objects. The object database can be extended dynamically as desired, which ensures a maximum functionality. A plurality of modules with identical or similar functionalities is also permitted. The data structures for the mathematical description of the integrated model are encapsulated in the individual modules. A particularity of this concept is the inter-module hierarchical combination of the data which, in turn, are administered by a separate database, analogously to the object modules. This makes possible the model-determined access to data from other modules. The modules of the simulation kernel must be generated by a master program and combined in the desired order.

To administer the various input parameters, a tie of the compound and organism parameters with the database was first realized. In addition (or else as the only alternative), a graphic user interface (GUI) ensures that all of the relevant model parameters (a) are visible to the user and (b) can be edited by the user. Modifying individual data in a module results in the testing of the dependencies in the remaining modules, in accordance with the data hierarchy, via predefined messages. Testing the data dependencies is an essential component of the model generator since it ensures that the model is correct at any given point in time. The result curves and, optionally, typical pharmacokinetic parameters which are derived from these curves (for example area under the curve, maximum concentration, time of maximum concentration, half-life, and the like) or pharmacodynamic parameters (for example intensity and duration of action) are indicated via the GUI.

The particular advantage of the present invention is the flexibility with which different and complex models can be generated in a simple manner. The examples which follow are intended to illustrate this fact with reference to figures. They show relevant scenarios of differing complexity. However, the examples are not to be understood as limiting the applicability of the present invention.

### BRIEF DESCRIPTION OF THE DRAWINGS

The figures show:

FIG. 1 schematic representation of a four-compartment mammalian model

FIG. 2 schematic representation of a physiological model of the mammalian body as a whole

FIG. 3 schematic representation of an organ in a model of the body as a whole

FIG. 4 schematic representation of a physiological model for caterpillars

FIG. 5 schematic representation of a physiological plant model

FIG. 6 schematic representation of the simplest model scenario: the model consists of the modules “compound”, “administration”, “organism”, “DEQ builder” and “integrator”. An “action module” can optionally be taken into consideration.

FIG. 7 schematic representation of the linking of the simulation model to databases and the graphic user interface.

FIG. 8 schematic representation of a model for multiple administration

FIG. 9 schematic representation of a model for a substance which enters the enterohepatic circulation

FIG. 10 schematic representation of a model for describing the interaction between two substances

FIG. 10a schematic representation of of a model for describing an active metabolite acting in parallel with the originally administered prodrug

FIG. 11 schematic representation of a model of two interacting organisms. Examples of such a scenario are the mother-foetus model and the combined insect-plant model.

FIG. 12 the concentration-time curve of a substance in various organs of a rat

FIG. 13 the concentration-time curve of a substance in the plasma of various mammals

FIG. 14 the concentration-time curve of a substance in various organs of a human

FIG. 15 the concentration-time curve of a substance in the plasma of a human following peroral administration

### EXAMPLES

This section describes preferred embodiments of the organisms to be described (mammal, insect and plant), and their incorporation into the dynamic simulation model is subsequently shown with reference to examples.

### Examples of Various Organism Modules

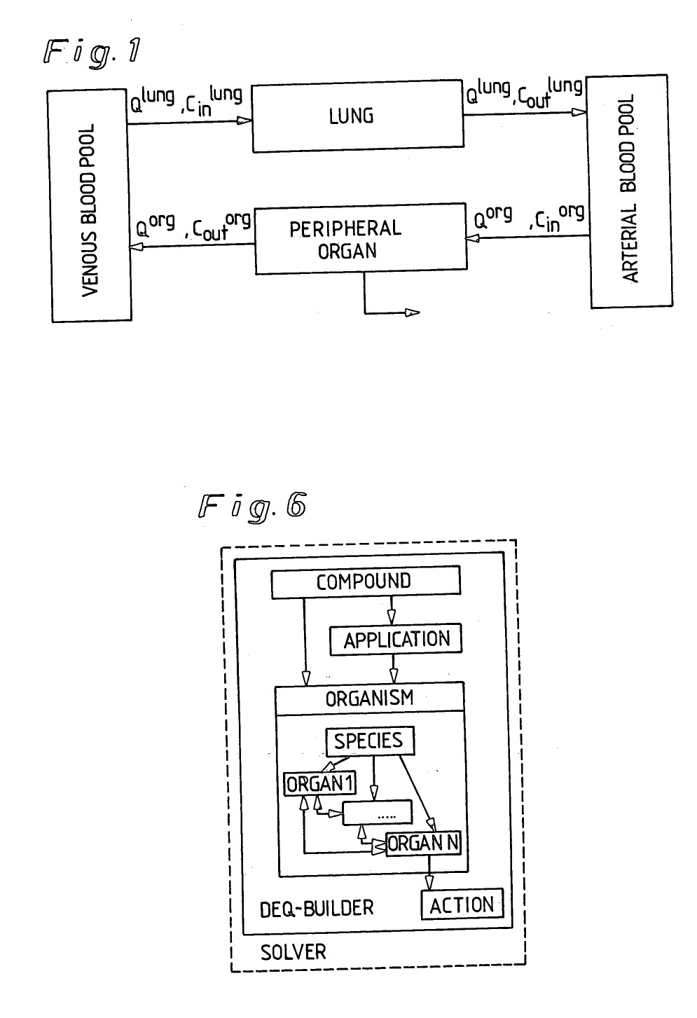

FIG. 1 shows a greatly simplified four-compartment model for a mammalian body. Here, the mammalian body consists only of the venous and arterial blood pools, the lungs and a further peripheral compartment via which the substance is eliminated metabolically. The peripheral compartment is provided with the substance (blood concentration Cⁱⁿ_{org}) via the arterial blood flow (blood flow rate Q_{org}). Following interaction in the compartment, the venous blood (concentration of the substance C^{out}_{org}) flows into the lungs, where the blood circulation is completed (Q_{lungs}=Q_{org}).

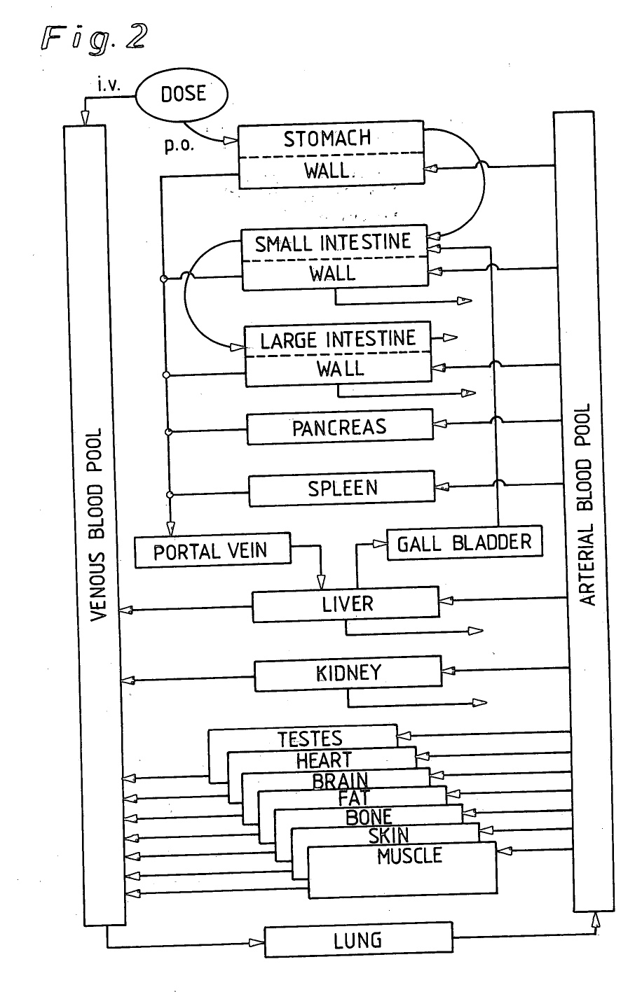

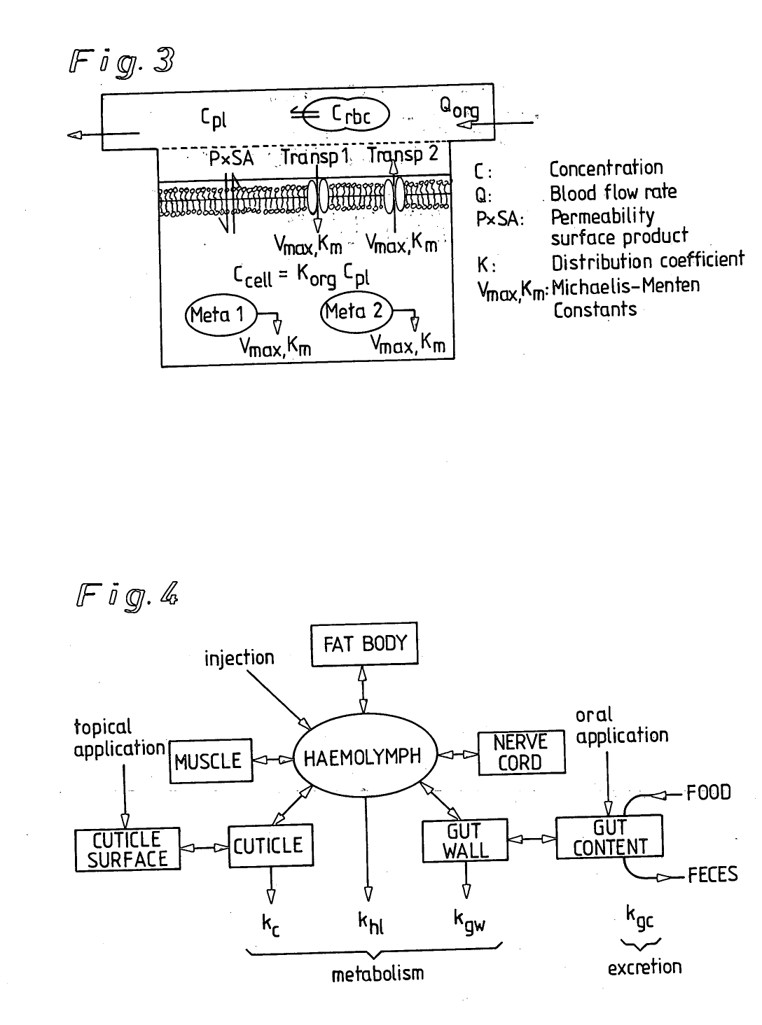

In a preferred embodiment of the invention, the mammalian model shown in FIG. 2 is used as the organism module. Here, the mammalian body likewise consists of the arterial and the venous blood pool, a lung compartment and the following peripheral organs: liver, kidney, muscle, bone, skin, fat, brain, stomach, small intestine, large intestine, pancreas, spleen, gall bladder and testes. This embodiment is referred to hereinbelow as a “model of the body as a whole”. Each organ in this model of a body as a whole is, in turn, divided into a plurality of subcompartments, each of which represents the vascular space (consisting of plasma and the red blood cells), the interstitial and the intracellular space (FIG. 3). The plasma and the interstitial space are treated as being in equilibrium. The transport of substances between the interstitial space and intracellular space is described as a passive diffusion process which follows the first-order kinetic, or as an active transport process which is modelled by a saturable Michaelis-Menten kinetic. Analogously, one or more Michaelis-Menten terms, which take into account the metabolic degradation of the substance, exist in each intracellular subcompartment.

The resulting system of coupled differential equations has the following form: three differential equations for the subcompartments “plasma” (pl), “red blood cells” or “blood cells” (bc) and “interior of the cell” (cell) exist for each peripheral organ superscript “org”. The model of the body as a whole comprises the following peripheral organs: lungs, stomach, small intestine, large intestine, pancreas, spleen, liver, kidney, brain, heart, muscle, bone, skin, fat and testes. The following

MASS BALANCE EQUATION results for the plasma and the interstitial space (int) for each organ:

| | MEANING |
|---|---|
| $[ {f}_{vas}^{org} ( 1 - HCT ) + {f}_{int}^{org} ] {V}^{org} \frac{ⅆ {C}_{pl}^{org}}{ⅆ t} = {Q}_{pl}^{org} ( {C}_{pl}^{art} - {C}_{pl}^{org} )$ | Intercompartmental flow term |
| $- \frac{{PA}_{bc}^{org}}{{K}_{pl}} ( {C}_{pl}^{org} - \frac{{C}_{bc}^{org}}{{K}_{bc}} )$ | Diffusive mass transport to the red blood cells |
| $- \frac{{PA}^{org}}{{K}_{pl}} ( {C}_{pl}^{org} - \frac{{C}_{cell}^{org}}{{K}^{org}} )$ | Diffusive mass transport into the interior of the cell |
| $- \frac{{V}_{max , in}^{org} {C}_{pl}^{org} / {K}_{pl}}{{K}_{m , in}^{org} + {C}_{pl}^{org} / {K}_{pl}}$ | Active transport term into the interior of the cell (influx) |
| $+ \frac{{V}_{max , ex}^{org} {C}_{cell}^{org} / ( {K}^{org} {K}_{pl} )}{{K}_{m , ex}^{org} + {C}_{cell}^{org} / ( {K}^{org} {K}_{pl} )}$ | Active transport term from the interior of the cell (efflux) |

The following MASS BALANCE EQUATION results for the red blood cells:

| | MEANING |
|---|---|
| ${f}_{vas}^{org} HCT {V}^{org} \frac{ⅆ {C}_{bc}^{org}}{ⅆ t} = {Q}_{bc}^{org} ( {C}_{bc}^{art} - {C}_{bc}^{org} )$ | Intercompartmental flow term |
| $+ \frac{{PA}_{bc}^{org}}{{K}_{pl}} ( {C}_{pl}^{org} - \frac{{C}_{bc}^{org}}{{K}_{bc}} )$ | Diffusive mass transport to the red blood cells |

The interior of the cell is described by means of the following

| MASS BALANCE EQUATION | MEANING |
|---|---|
| ${f}_{cell}^{org} {V}^{org} \frac{ⅆ {C}_{cell}^{org}}{ⅆ t} = \frac{{PA}^{org}}{{K}_{pl}} ( {C}_{pl}^{org} - \frac{{C}_{cell}^{org}}{{K}^{org}} )$ | Diffusive mass transport into the interior of the cell |
| $+ \frac{{V}_{max , in}^{org} {C}_{pl}^{org} / {K}_{pl}}{{K}_{m , in}^{org} + {C}_{pl}^{org} / {K}_{pl}}$ | Active transport term into the interior of the cell (influx) |
| $- \frac{{V}_{max , ex}^{org} {C}_{cell}^{org} / ( {K}^{org} {K}_{pl} )}{{K}_{m , ex}^{org} + {C}_{cell}^{org} / ( {K}^{org} {K}_{pl} )}$ | Active transport term from the interior of the cell (efflux) |
| $+ \frac{{V}_{max , M1}^{org} {C}_{cell}^{org} / ( {K}^{org} {K}_{pl} )}{{K}_{m , M1}^{org} + {C}_{cell}^{org} / ( {K}^{org} {K}_{pl} )}$ | Metabolic degradation (enzyme 1) |
| $+ \frac{{V}_{max , M2}^{org} {C}_{cell}^{org} / ( {K}^{org} {K}_{pl} )}{{K}_{m , M1}^{org} + {C}_{cell}^{org} / ( {K}^{org} {K}_{pl} )}$ | Metabolic degradation (enzyme 2) |
| $- {CL}^{org} {C}_{cell}^{org}$ | First-order degradation (for example in the liver or the kidneys) |

The lungs (org=lng) is described analogously; however, the inward flow does not originate from the arterial blood pool (art), but from the venous blood pool (ven):

| MASS BALANCE EQUATION | MEANING |
|---|---|
| $[ {f}_{vas}^{ln g} ( 1 - HCT ) + {f}_{int}^{ln g} ] {V}^{ln g} \frac{ⅆ {C}_{pl}^{ln g}}{ⅆ t} = {Q}_{pl}^{ln g} ( {C}_{pl}^{ven} - {C}_{pl}^{ln g} )$ | Blood flow is reversed in comparison with the remaining organs! |
| ± . . . | Analogous to the other organs |
| ${f}_{vas}^{ln g} HCT {V}^{ln g} \frac{ⅆ {C}_{bc}^{ln g}}{ⅆ t} = {Q}_{bc}^{ln g} ( {C}_{bc}^{ven} - {C}_{bc}^{ln g} )$ | Blood flow is reversed in comparison with the remaining organs! |
| ± . . . | Analogous to the other organs |

The concentrations flowing outwardly from the organs combine to form the venous blood pool. The resulting plasma concentration is the result of the blood-flow-weighted mean of the individual organ concentrations:

${XV}^{ven} \frac{ⅆ {C}_{pl / bc}^{ven}}{ⅆ t} = {Q}_{pl / bc}^{ln g} ( {\sum }_{org} {Q}_{pl / bc}^{org} {C}_{pl / bc}^{org} / {\sum }_{org} {Q}_{pl / bc}^{org} - {C}_{pl / bc}^{ven} )$

| MASS BALANCE EQUATION | MEANING |
|---|---|
| $∓ \frac{{PA}_{bc}^{ven}}{{K}_{pl}} ( {C}_{pl}^{ven} - \frac{{C}_{bc}^{ven}}{{K}_{bc}} )$ | Diffusive mass transport between plasma (−) and red blood cells (+) |
| $- {CL}_{ven} {C}_{pl}^{ven}$ | Plasma clearance (if appropriate) |
| $+ \frac{ⅆ {IV}_{pl}}{ⅆ t}$ | Input function for application to the venous blood pool |

$X = \{ \begin{matrix}1 - HCT for plasma \\ HCT for red blood cells\end{matrix}$

Finally, the arterial blood pool is described by:

${XV}^{art} \frac{ⅆ {C}_{pl / bc}^{art}}{ⅆ t} = {Q}_{pl / bc}^{ln g} ( {C}_{pl / bc}^{ln g} - {C}_{pl / bc}^{art} )$

| MASS BALANCE EQUATION | MEANING |
|---|---|
| $∓ \frac{{PA}_{bc}^{art}}{{K}_{pl}} ( {C}_{pl}^{art} - \frac{{C}_{bc}^{art}}{{K}_{bc}} )$ | Diffusive mass transport between plasma (−) and the red blood cells (+) |

$X = \{ \begin{matrix}1 - HCT for plasma \\ HCT for red blood cells\end{matrix}$

As an alternative to, or in combination with, the intravenous application described herein, it is also possible to simulate uptake via the gastrointestinal mucosa following peroral administration. The solutions of this equation system give the concentration-time relationships for all of the compartments present in the model.

To describe a pharmacological activity, it is furthermore possible to link the concentration-time relationship in the compartment which contains the biological target of the active compound with a pharmacodynamic effect. Examples of typical effect functions are:

Hyperbolic or sigmoidal Emax models:

$$Effect = {E}_{0} + \frac{{E}_{max} {C}_{x}^{γ}}{{EC}_{50}^{γ} + {C}_{x}^{γ}}$$

Effect=parameter of the pharmacological activity (time-dependent)

E₀=base value of the parameter of the pharmacological activity

Eₘₐₓ=maximum pharmacological activity

EC₅₀=concentration at which 50% of the maximum effect has been obtained

Cₓ=concentration at the site of action (time-dependent)

γ=form parameter

Power functions: Effect=E₀+β Cₓ^{γ}, or Log linear models:
Effect=E₀+βLn(Cₓ)

Effect=parameter of the pharmacological activity (time-dependent)

E₀=base value of the parameter of the pharmacological activity

β=parameter for the increase of the effect as a function of the concentration

Cₓ=concentration at the site of action (time-dependent)

γ=form parameter

Active compound interaction models such as, for example, partial or total antagonism, and the like

Combinations of the abovementioned models with the aid of which for example centres of multiple activity or receptor-transducer interactions can be described.

In a further preferred embodiment of the invention, the organism module represents the anatomy of an insect, in particular of a caterpillar (FIG. 4). The body of the caterpillar consists of the following compartments: haemolymph as central compartment, cuticle, muscle, fat body, nerve system and gut wall as peripheral compartments, and the compartments cuticle surface and gut content, via which substances can be exchanged with the environment. The intercompartmental mass transport, in turn, can take the form of passive transport via diffusion or as an active process with the aid of transporters. The resulting differential equation system has been described in the appendix of the as yet unpublished German Patent Application with file reference 10256315.2.

The plant model of FIG. 5 constitutes a further preferred embodiment. In accordance with the typical physiology of a plant, the model describes the roots, stems and leaves of a plant. Each of these compartments, in turn, consists of three subcompartments which represent the vacuole, the symplast and the apoplast. These subcompartments are separated from one another by membranes. Like in the above-described organisms, these membranes can again be permeated by passive diffusion or by means of active transport. Moreover, the subcompartments are characterized by different pH values, which greatly influence in particular the distribution behaviour of acids and bases. The plant has two translocation pathways between the compartments: The xylem flux in the apoplast flows from the root towards the leaves, while the phloem flux, in turn, translocates substances in the symplast from the leaves to the roots.

### Examples of Model Structures

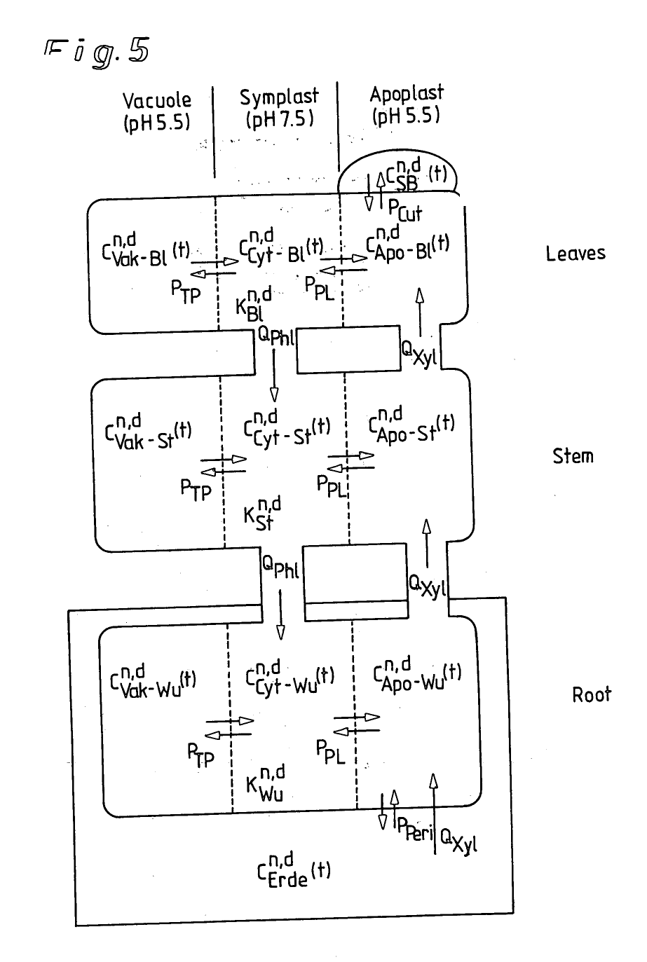

The simplest model structure (FIG. 6)

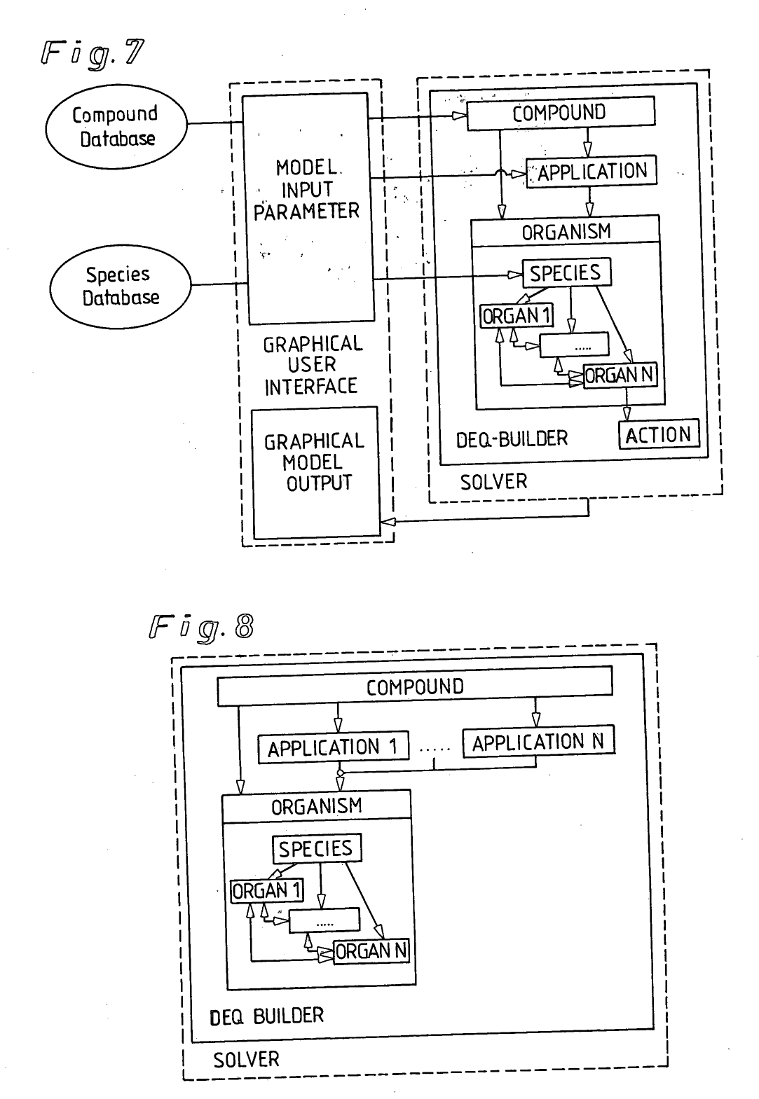

FIG. 6 shows the simplest model structure. The complete model consists of the modules “compound”, “administration”, “organism”, “DEQ builder” and “integrator”. An “action module” can optionally be taken into consideration. FIG. 7 is a schematic representation of the linking to one or more databases which may comprise, for example, the compound parameters or the physiological and anatomical information of the organism in question.

Multiple administration (FIG. 8)

The repeated administration of an active compound to one and the same organism is a realistic scenario for a multiplicity of pharmaceutical compounds which must be taken at regular intervals over a prolonged period, such as, for example, anti-infective active compounds. This model is an example of how long-term effects such as, for example, the accumulation of the active compound in the individual organisms, can be simulated.

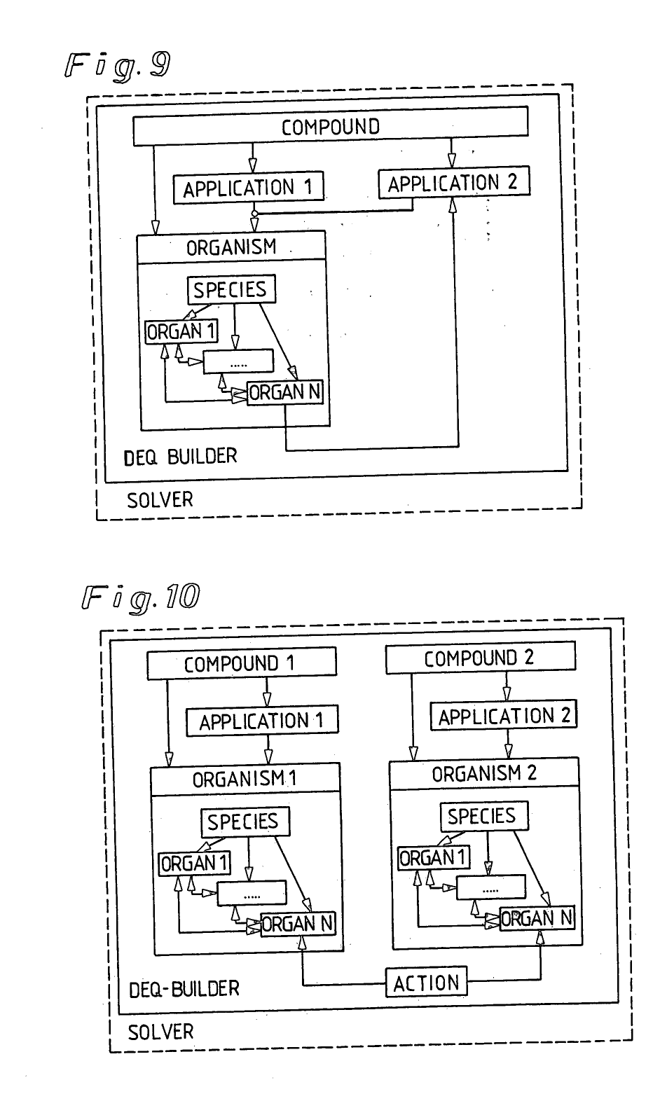

Enterohepatic circulation (FIG. 9)

A series of chemical compounds which were administered to mammals enter the enterohepatic circulation. In such a case, some of the compound which has been administered is, in the liver, secreted into the bile without modification and therein accumulated in the gall bladder (represented by ORGAN N in FIG. 9). When triggered by a chemical compound stimulus, for example by taking a meal, the gall bladder contracts and releases its contents—bile which, inter alia, also contains the active compound—into the duodenum. The compound can then be absorbed via the gut and thus re-enter the systemic circulation. In the model outlined in FIG. 8, this process can be described simply as a renewed intestinal application (APPLICATION 2, characterized by a time lag) of part of the original compound.

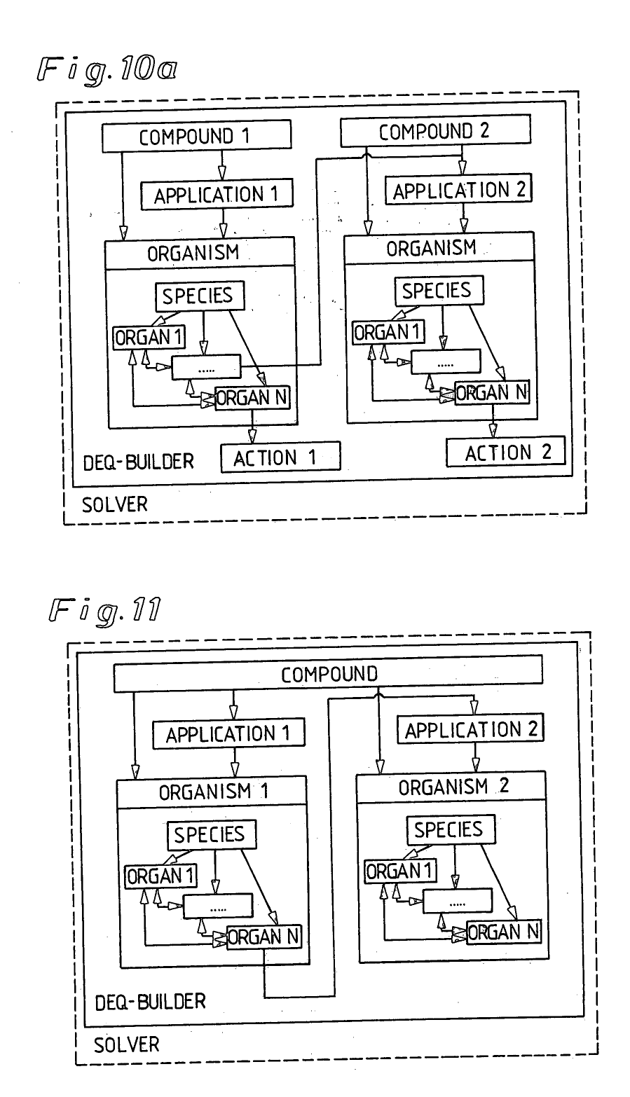

Drug-drug interaction (FIG. 10)

Interactions of a plurality of compounds (in the present example: two compounds) between one another are very important. By way of a model, this case can be shown as follows: ORGANISM 1 and 2 are defined via identical physiological parameters since they represent one and the same organism. Two different compounds are administered separately; the route, timing and duration of administration may differ for the two compounds. The actual interaction of the two compounds is defined in an action module which links the organs ORGAN N in the organisms 1 and 2. For example, the interaction can be a competitive inhibition or any other biochemical interaction which is feasible.

Active metabolite/prodrug (FIG. 10a)

In many cases a metabolic product which is also pharmacodynamically active (an “active metabolite”) is formed for example in the intestine or the liver by metabolization of the originally administered parent substance. Thus two substances circulate simultaneously within the organism. This case is illustrated in FIG. 10a. The starting COMPOUND 1 originally administered by APPLICATION 1 is converted within one of the organs of the body (e.g. in the wall of the intestine or the liver) at a specific rate to form COMPOUND 1. At the same rate the metabolite COMPOUND 2 is formed in the organ concerned. This process is illustrated by the module for APPLICATION 2. Both substances can act on one and the same or on different targets (ACTION 1 and 2). One special example of such a phenomenon is the so-called prodrug concept in which a starting substance (a prodrug) which is initially inactive is administered and only converted into the active substance by metabolization within the body (i.e. ACTION 1=0).

Mother-foetus model (FIG. 11)

The mother-foetus model in FIG. 11 is an example in which two different, but coupled, organisms are modelled simultaneously. ORGANISM 1 represents the mother, while ORGANISM 2 represents the foetus. The blood circulation of the foetus is connected to the blood circulation of the mother via the placenta (in the present case represented by ORGAN N in ORGANISM 1). In this example, it is highly important that the physiological parameters, in particular the organ volumes and blood flow rates of the foetus, can be functions of time in order to be able to take into consideration the precise gestation age and the growth of the foetus in the womb.

Combined plant/insect models (FIG. 11)

Like the mother-foetus model, the plant/insect model is also a combination of two coupled organisms; however, the physiology of the organisms differs in the present case. ORGANISM 1 describes a plant which takes up a compound which has been applied, for example an insecticide, via the foliar cuticles or via the roots. Following distribution in all of the organism, this compound is also available in the remaining compartments. Feeding from a leaf, which constitutes a specific compartment in the plant organism, allows the compound to be taken up by an insect (ORGANISM 2), where it is distributed; finally, it achieves its insecticidal activity in the target organ, for example the nervous system of the caterpillar.

Further Combinations of the Above-Described Examples

Combinations of the above-described examples are likewise very important. For example, the multiple administration of two or more interactive substances in a mother-foetus model can be used for the early assessment of the toxicological risk to the foetus.

Simulation results for the model of the mammalian body as a whole

In the following text, the steps required for performing a simulation will be shown by way of example. First, the compound-dependent characteristics must be determined. A compound X with the following characteristics serves as an example:

TABLE 1
Compound-dependent parameters for the compound X

| Parameter | Value | Unit |
|---|---|---|
| Lipophilicity (LogMA) | 2.7 | —/— |
| Unbound plasma fraction | 0.25 | —/— |
| Liver clearance | 1 | ml/min/kg |
| Dose | 1 | mg/kg |

The organ distribution coefficients can now be calculated from the lipophilicity (MA=10\hat{ }LogMA) and the unbound plasma fraction (fᵤ) with the aid of published equations [M. Härter, J. Keldenich, W. Schmitt: Estimation of physicochemical and ADME parameters, Ch. 26 in: Combinatorial Chemistry—A Practical Handbook, Part IV. Eds. K. C. Nicolau et al., Wiley VCH, Weinheim, 2002]. In the present example, the following values result for the organ distribution coefficients:

TABLE 2
Distribution coefficients [8] resulting from the values of Table 1.
Organ/plasma distribution coefficient

| | |
|---|---|
| Stomach | 8.34 |
| Small intestine | 8.34 |
| Large intestine | 8.34 |
| Pancreas | 10.57 |
| Spleen | 2.74 |
| Liver | 9.35 |
| Kidney | 7.19 |
| Lungs | 1.97 |
| Brain | 14.21 |
| Heart | 13.28 |
| Muscle | 2.33 |
| Bone | 34.45 |
| Skin | 13.49 |
| Fat | 100.42 |
| Testes | 4.42 |

Moreover, the physiological parameters such as organ volumes, organ blood flow rates and composition must be known with regard to the vascular, interstitial and cellular space. These parameters are likewise described in the literature. For example, the values listed in Tables 3 to 5 are found for the species mouse, rat, dog and man:

TABLE 3
Organ volumes for mouse, rat, dog and man (literature data)

| Organ volumes [ml] | MOUSE | RAT | DOG | MAN |
|---|---|---|---|---|
| Venous blood pool | 0.128 | 6.8 | 0.8 | 250 |
| Arterial blood pool | 0.073 | 2.9 | 0.8 | 140 |
| Lungs | 0.201 | 2.2 | 145 | 670 |
| Stomach | 0.1 | 1.1 | 40 | 150 |
| Small intestine | 0.2 | 11.1 | 140 | 640 |
| Large intestine | 0.2 | 11.1 | 140 | 370 |
| Pancreas | 0.13 | 1.3 | 10 | 100 |
| Spleen | 0.13 | 1.3 | 10 | 180 |
| Liver | 0.941 | 10 | 366 | 1710 |
| Gall bladder | 0.12 | 1.2 | 10 | 20 |
| Kidney | 0.449 | 7 | 154 | 720 |
| Brain | 0.336 | 1.671 | 90.4 | 1486 |
| Heart | 0.5 | 1.2 | 90 | 330 |
| Muscle | 11.74 | 110.1 | 6502 | 30200 |
| Bone | 2.775 | 28.2 | 2584 | 12060 |
| Skin | 5.16 | 43.4 | 647 | 3020 |
| Fat | 1.51 | 14.2 | 2670 | 10060 |
| Testes | 0.001 | 2.5 | 5 | 35 |
| Organs in total | 24.694 | 257.271 | 13605 | 62141 |

TABLE 4
Organ blood flow rates for mouse, rat, dog and man
(literature data)

| Blood flow rate [ml/min] | MOUSE | RAT | DOG | MAN |
|---|---|---|---|---|
| Stomach | 0.5 | 1.22 | 40 | 60 |
| Small intestine | 0.5 | 7.02 | 400 | 600 |
| Large intestine | 0.5 | 3.82 | 160 | 240 |
| Pancreas | 0.005 | 0.51 | 40 | 60 |
| Spleen | 0.063 | 0.63 | 120 | 180 |
| Liver | 2.25 | 5.5 | 310 | 390 |
| Kidney | 0.67 | 14.6 | 243 | 1133 |
| Brain | 0.11 | 1.1 | 145 | 700 |
| Heart | 0.039 | 3.92 | 180 | 240 |
| Muscle | 0.33 | 7.2 | 118 | 550 |
| Bone | 0.007 | 1.6 | 35 | 167 |
| Skin | 0.02 | 4.8 | 10 | 50 |
| Fat | 0.002 | 1.8 | 3 | 300 |
| Testes | 0.0002 | 0.48 | 0.66 | 2.6 |
| Lungs (= total) | 4.996 | 53.72 | 1805 | 4670 |

TABLE 5
Organ composition of mammals (literature data)

| Proportion by volume | f_vas | f_int | f_cell |
|---|---|---|---|
| Fatty tissue | 0.010 | 0.135 | 0.855 |
| Brain | 0.037 | 0.004 | 0.959 |
| Gastrointestinal tract | 0.032 | 0.100 | 0.868 |
| Heart | 0.262 | 0.100 | 0.638 |
| Kidney | 0.105 | 0.200 | 0.695 |
| Liver | 0.115 | 0.163 | 0.722 |
| Lungs | 0.626 | 0.188 | 0.186 |
| Muscle | 0.026 | 0.120 | 0.854 |
| Bone | 0.041 | 0.100 | 0.859 |
| Skin | 0.019 | 0.302 | 0.679 |
| Pancreas | 0.180 | 0.120 | 0.700 |
| Spleen | 0.282 | 0.150 | 0.568 |
| Testes | 0.140 | 0.069 | 0.791 |

The following text shows simulation results which have been achieved with the model of the body as a whole as shown in FIG. 3. As the simplest borderline case, it was assumed that the transport into the organs is blood-flow-limited (i.e. PA^{org}→∝).

FIG. 12 shows the resulting organ concentrations in the rat following intravenous administration of 1 mg/kg of compound X in the form of a bolus.

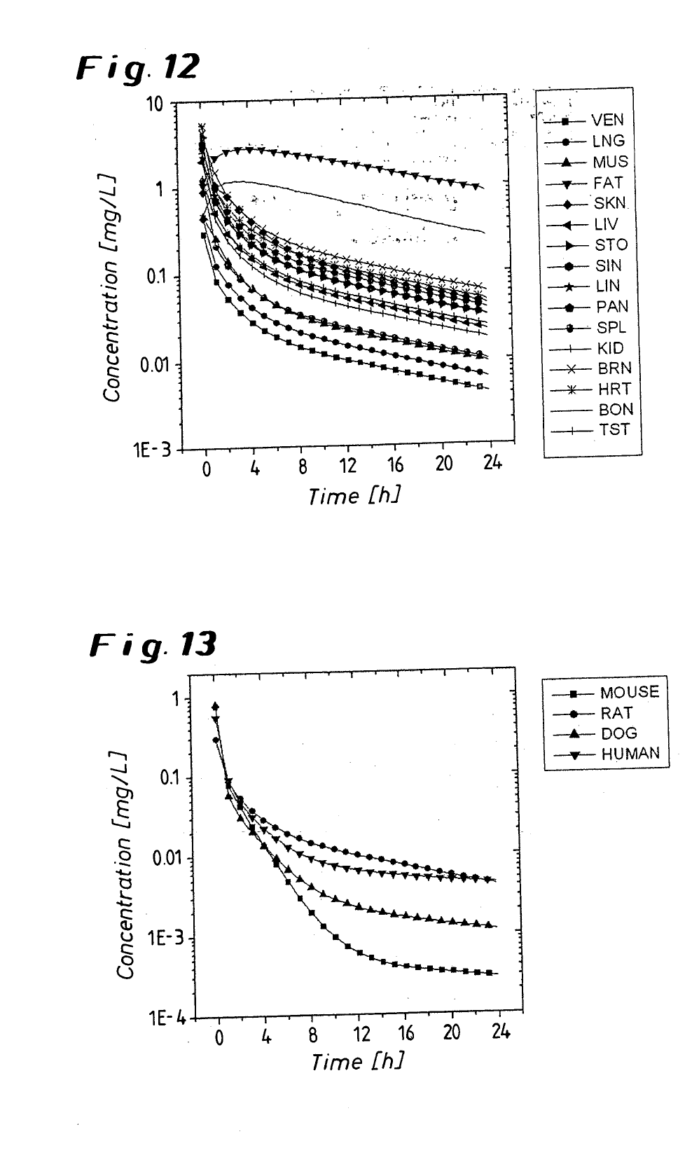

FIG. 13 shows the concentration-time curve in the plasma simulated in the four different species following intravenous administration of 1 mg/kg of compound X in the form of a bolus.

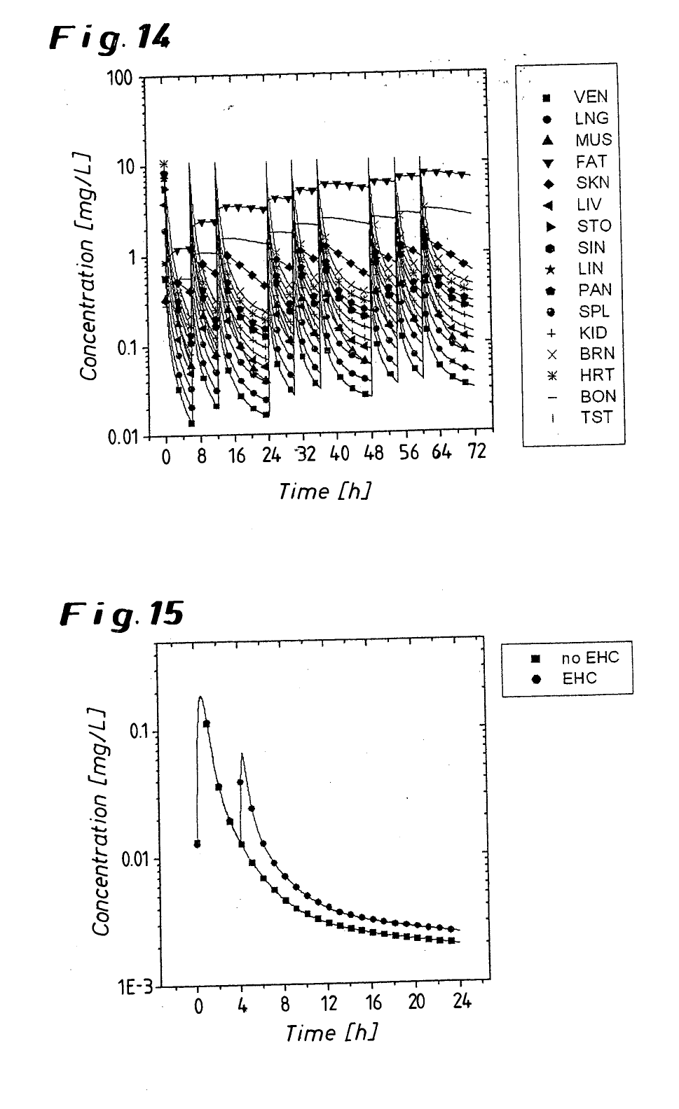

FIG. 14 shows the concentration-time curves in the various organs of a human following multiple intravenous administration over 3 days of 1 mg/kg of compound X in the form of a bolus (regimen: 6 h-6 h-12 h, calculated in accordance with the model in FIG. 8).

FIG. 15 shows the concentration-time curve in the plasma following peroral administration of 1 mg/kg of the compound X in man without enterohepatic circulation and with enterohepatic circulation, calculated in accordance with the model in FIG. 9 (assuming that clearance with the bile amounts to 25% of the total clearance).

The foregoing is only a description of a non-limiting number of embodiments of the present invention. It is intended that the scope of the present invention extend to the full scope of the appended issued claims and their equivalents.

1. Method for determining the interaction of one or more active compounds administered to an organism, said method comprising the following steps:
A) selecting at least one compound and analyzing the compound with respect to one or more characteristics selected from the group consisting of lipophilicity, solubility, protein binding, molecular weight, molecular volume, pKa value in the case of acids or bases, metabolic degradation rate and kinetic constants of the compound's active transporters,
B) selecting one or more anatomical and physiological compartments of the organism in question as follows:
i) in the case of mammals or insects, the selecting is from the group consisting of lungs, peripheral organs, blood vessels, arteries, veins, and blood fluid, and
ii) in the case of plants, the selecting is from the group consisting of xylem, phloem, root, leaf and stem,
C) preparing a description of one or more of the compartments in a computer program with regard to one or more transport processes selected from the group consisting of transport from and to the compartments, degradation processes, and active transport processes, and wherein the description comprises mass transport equations and kinetic reaction equations,
D) describing the transport processes and/or the degradation processes of the administered active compound in the organism by mass transport equations and kinetic reaction equations,
E) combining the equations in steps C) and D), to provide a numerical calculation of the resulting system of coupled differential equations, and
F) determining the concentration-time curve of the active compound in the selected compartments.

2. The method according to claim 1, wherein the organism(s) are selected from the group consisting of humans, monkeys, dogs, pigs, rats, mice, insects and plants.

3. The method according to claim 1, wherein the active compound is applied via a route selected from the group consisting of intravenous, oral, topical, subcutaneous, intraperitoneal, inhalative via the nose or the lungs, and intracerebrovascular in the case of mammals, or intrahaemolymphatic, topical or oral via feeding or gavage in the case of insects, or in the form of a spray mixture with foliar uptake or uptake via the roots in the case of plants.

4. The method according to claim 1, wherein the physiological parameters which describe the organisms are time-dependent parameters.

5. The method according to claim 1, wherein one or more of the parameters are taken from a database which is linked to a computer system.

6. The method according to claim 1, wherein the physiological and anatomical parameters are varied within a predetermined statistical variation by means of random numbers.

7. The method according to claim 1, wherein a computing program comprises a hierarchical model architecture consisting of modules with different functionalities, wherein at least two of the modules comprise either chemical or biological characteristics of the compound, physiological and/or anatomical information on the organism, information on the mode of the compound's application, or the type of transport and/or metabolic process the compound is undergoing.

8. The method of claim 7, wherein an integrated model simulates drug-drug interactions in a mother-foetus model, or combined insect-plant model.

9. The method according to claim 1, wherein a multiplicity of models of organisms are combined in an integrated model.

10. The method according to claim 1, wherein the active compound is administered by a route selected from the group consisting of intravenous, peroral and subcutaneous administration.
